(19) United States
(12) Patent Application Publication
(10) Pub. No.: US 2025/0122141 A1
(43) Pub. Date: Apr. 17, 2025

(54) CHEMICAL RECYCLING DEVICE AND CHEMICAL RECYCLING MOLDING SYSTEM

(71) Applicant: SUMITOMO HEAVY INDUSTRIES, LTD., Tokyo (JP)

(72) Inventors: Takashi SUZUKI, Yokosuka-shi (JP); Yoshiko SHISHIDO, Yokosuka-shi (JP); Mayuka NOMURA, Yokosuka-shi (JP); Takayuki MENJO, Yokosuka-shi (JP); Kouji SEIKE, Chiba-shi (JP)

(21) Appl. No.: 19/002,483

(22) Filed: Dec. 26, 2024

Related U.S. Application Data
(63) Continuation of application No. PCT/JP2023/025577, filed on Jul. 11, 2023.

(30) Foreign Application Priority Data
Jul. 13, 2022 (JP) 2022-112699
Oct. 19, 2022 (JP) 2022-167799

(51) Int. Cl. C07C 63/26; B29C 45/00; B29K 67/00; B29K 105/00; B29K 105/26; B29L 31/00

(52) U.S. Cl. CPC C07C 63/26; B29C 45/0001; B29K 2067/003; B29K 2105/0067; B29K 2105/26; B29L 2031/7158; C07C 2601/16

(57) ABSTRACT

A chemical recycling device includes a depolymerization reaction tank that decomposes a first molding product made of a polymer into a depolymerized product through a depolymerization reaction, a polymerization reaction tank that synthesizes the depolymerized product into the polymer through a polymerization reaction, and a polymer supply unit that supplies the polymer synthesized in the polymerization reaction tank to a molding machine that molds a second molding product.

### CROSS-REFERENCE TO RELATED APPLICATIONS

This is a bypass continuation of International PCT Application No. PCT/JP2023/025577, filed on Jul. 11, 2023, which claims priority to Japanese Patent Application No. 2022-112699, filed on Jul. 13, 2022 and Japanese Patent Application No. 2022-167799, filed on Oct. 19, 2022, which are incorporated by reference herein in their entirety.

### BACKGROUND

### Technical Field

Certain embodiments of the present invention relate to a chemical recycling device and the like.

### Description of Related Art

The related art discloses discloses a method of manufacturing polyethylene terephthalate (PET) flakes, which are raw materials for a new PET bottle, by pulverizing a PET bottle for recycling the PET bottle. Specifically, mechanical recycling for obtaining PET flakes by means of solid phase polymerization or the like after the pulverized PET bottle is heated and melted, and chemical recycling for obtaining PET flakes by means of a repolymerization reaction after the pulverized PET bottle is decomposed into an intermediate such as bis(2-hydroxyethyl) terephthalate (BHET) or a depolymerized product by a depolymerization reaction are known.

### SUMMARY

According to an embodiment of the present invention, there is provided a chemical recycling device including a depolymerization reaction tank that decomposes a first molding product made of a polymer into a depolymerized product through a depolymerization reaction, a polymerization reaction tank that synthesizes the depolymerized product into the polymer through a polymerization reaction, and a polymer supply unit that supplies the polymer synthesized in the polymerization reaction tank to a molding machine that molds a second molding product.

According to another embodiment of the present invention, there is also provided a chemical recycling device. This device includes a polymerization reaction tank that synthesizes a depolymerized product obtained by decomposing a first molding product made of a polymer through a depolymerization reaction into the polymer through a polymerization reaction, and a polymer supply unit that supplies the polymer synthesized in the polymerization reaction tank to a molding machine that molds a second molding product.

According to still another embodiment of the present invention, there is also provided a chemical recycling device. This device includes a polymer supply unit that supplies a polymer, which is synthesized through a polymerization reaction from a depolymerized product obtained by decomposing a first molding product made of the polymer through a depolymerization reaction, to a molding machine that molds a second molding product.

### BRIEF DESCRIPTION OF THE DRAWINGS

FIG. 1 schematically shows a configuration of a chemical recycling molding system according to one embodiment.

FIG. 2 schematically shows a polymerization reaction and a depolymerization reaction of PET.

FIG. 3 shows a modification example of a by-product removal device.

FIG. 4 schematically shows a configuration of a chemical recycling molding system according to another embodiment.

FIG. 5 schematically shows a configuration of a chemical recycling molding system according to still another embodiment.

FIG. 6 schematically shows a depolymerization reaction.

FIG. 7 schematically shows a configuration of a chemical recycling molding system according to still another embodiment.

FIG. 8 schematically shows a first example of a mechanical mixing unit.

FIG. 9 schematically shows the first example of the mechanical mixing unit.

FIG. 10 schematically shows a second example of the mechanical mixing unit.

FIG. 11 schematically shows the second example of the mechanical mixing unit.

FIG. 12 schematically shows a third example of the mechanical mixing unit.

FIG. 13 schematically shows the third example of the mechanical mixing unit.

FIG. 14 shows a first modification example of the chemical recycling molding system.

FIG. 15 shows a second modification example of the chemical recycling molding system.

### DETAILED DESCRIPTION

In a case of manufacturing the PET flakes or PET pellets as described above (hereinafter, the PET flakes and the PET pellets are simply referred to as flakes and pellets, which also refer to the flakes and pellets that are not made of PET as a raw material), it is necessary to cool the PET in a molten state to manufacture the flakes or the pellets. In addition, in a case of manufacturing a new PET bottle or the like from the flakes or pellets, the flakes or pellets need to be heated and melted and then supplied to a molding machine such as an injection molding machine. The cooling during the manufacturing of such flakes or pellets and the heating of the flakes or pellets during the manufacturing of a new PET bottle or the like are substantially opposite thermal processes. The presence of the cooling process and the heating process relating to the flakes and the pellets, which do not have a substantial meaning in view of the purpose of recycling PET bottles or the like into PET bottles or the like, can be seen as wasting energy in the process of recycling aimed at conserving resources and saving energy.

It is desirable to provide a chemical recycling device or the like capable of recycling a molding product such as a PET bottle with less energy as compared with the related art.

In this aspect, the polymer resynthesized in the polymerization reaction tank is not made into flakes or pellets, and is supplied as it is to the molding machine by the polymer supply unit. Since the cooling process and the heating process related to the flakes or the pellets as in the related art are not required, the molding product such as a PET bottle can be recycled with less energy as compared with the related art.

Any combination of the above-described components or any expression of these components converted into a method, a device, a system, a recording medium, a computer program, or the like is also included in the present invention.

Hereinafter, an embodiment for carrying out the present invention (hereinafter, also referred to as an embodiment) will be described in detail with reference to the drawings. In the description and/or the drawings, identical or equivalent components, members, and processes are denoted by the same reference numerals, and overlapping description is omitted as appropriate. The scale or shape of each part that is shown in the drawings is conveniently set for simplicity and ease of description and is not limitedly interpreted unless otherwise specified. The embodiment is exemplary and does not limit the scope of the present invention in any way. All features to be described in the embodiment and combinations thereof are not necessarily essential to the present invention.

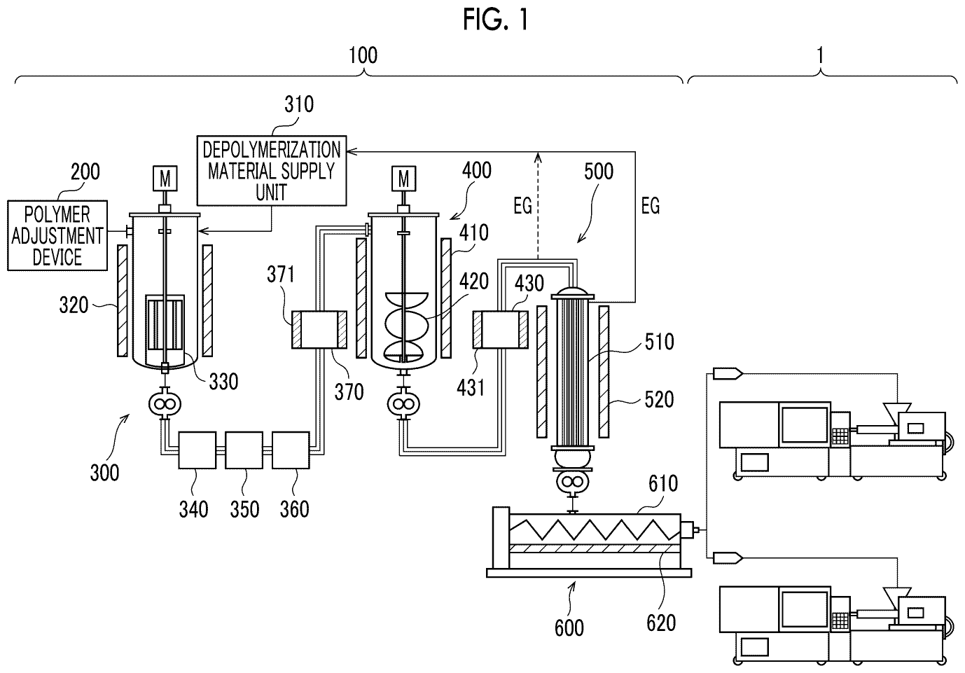

FIG. 1 schematically shows a configuration of a chemical recycling molding system according to one embodiment of the present invention. The chemical recycling molding system includes a chemical recycling device 100 and an injection molding machine 1. The chemical recycling device 100 includes a polymer adjustment device 200, a depolymerization reaction tank 300, a polymerization reaction tank 400, a by-product removal device 500, and a polymer supply unit 600. The number of injection molding machines 1 (two are schematically shown in FIG. 1), the number of polymer adjustment devices 200, the number of depolymerization reaction tanks 300, the number of polymerization reaction tanks 400, the number of by-product removal devices 500, and the number of polymer supply units 600 are optional. In particular, by increasing the number of injection molding machines 1 and polymerization reaction tanks 400, which typically have slower processing speeds or reaction rates than other processing units, the processing performance can be improved so that these processing units do not become serious bottlenecks.

The polymer adjustment device 200 adjusts a polymer such as PET that constitutes a first molding product such as a PET bottle for the depolymerization reaction tank 300 in a subsequent stage. Specifically, the polymer adjustment device 200 performs a process such as pulverizing, heating and melting, and mixing on the first molding product such as a PET bottle, and adjusts the polymer such as PET to a suitable state (phase, shape, size, and the like) for a depolymerization reaction in the depolymerization reaction tank 300. The first molding product may be any molding product other than a bottle, such as a sheet, a film, or a fiber. In addition, the polymer constituting the first molding product may be any polymer other than PET, such as polyester (including PET), polyamide, and polyurethane.

The depolymerization reaction tank 300 decomposes the polymer such as PET adjusted by the polymer adjustment device 200 into a depolymerized product through a depolymerization reaction. In a case where the polymer supplied from the polymer adjustment device 200 is PET, BHET, which is an intermediate, is obtained as the depolymerized product through the depolymerization reaction in the depolymerization reaction tank 300. The depolymerized product obtained in the depolymerization reaction tank 300 may contain a monomer of the polymer. In a case where the polymer is PET, the monomer is, for example, ethylene glycol, terephthalic acid, dimethyl terephthalate, or ethylene terephthalate.

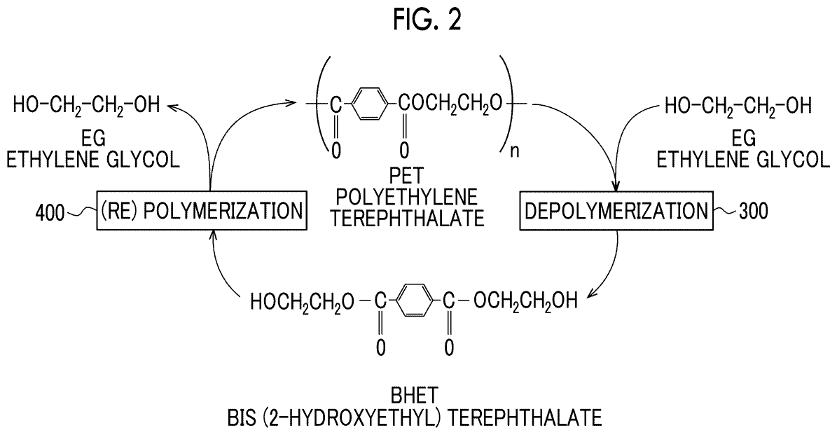

As schematically shown in FIG. 2, in the depolymerization reaction (300) of PET as a polymer, PET is decomposed by ethylene glycol (EG) as a depolymerization material supplied from a depolymerization material supply unit 310 (FIG. 1) to the depolymerization reaction tank 300, and BHET as a depolymerized product is obtained. In addition, instead of or in addition to the depolymerization material supply unit 310, EG may be supplied in the polymer adjustment device 200. In order to promote such a depolymerization reaction, an inside of the depolymerization reaction tank 300 is maintained at a suitable temperature for the depolymerization reaction by a heating unit 320 (FIG. 1) or a temperature maintaining unit that is provided in conjunction with the depolymerization reaction tank 300. A suitable temperature for the depolymerization reaction of the PET to the BHET in FIG. 2 is between 220° C. and 250° C., is preferably between 230° C. and 245° C., and is more preferably between 235° C. and 240° C. In addition, a suitable pressure for the depolymerization reaction of the PET to the BHET in FIG. 2 is between 0.3 MPa and 0.8 MPa, is preferably between 0.4 MPa and 0.6 MPa, and is more preferably between 0.45 MPa and 0.55 MPa. The pressure in the depolymerization reaction tank 300 is adjusted by a pump (not shown) or the like that is provided in conjunction with the depolymerization reaction tank 300.

A viscosity of a fluid in the depolymerization reaction tank 300 in which BHET having a smaller molecular weight than PET, which is a polymer, is generated is lower than a viscosity of a fluid in the polymerization reaction tank 400, which will be described later, in which PET having a larger molecular weight is generated. Therefore, a stirring blade 330 for stirring the fluid in the depolymerization reaction tank 300 to promote the depolymerization reaction is used for low viscosity. A propeller blade, a disc turbine blade, and a paddle blade are exemplified as the stirring blade 330 for low viscosity.

In the subsequent stage of the depolymerization reaction tank 300, foreign matter removal devices 340, 350, and 360 for removing foreign matter from the fluid mainly composed of BHET as the depolymerized product are provided.

A different resin removal device 340 removes a resin and/or a depolymerized product different from a target resin such as PET by using principles of floating separation and sedimentation removal. A colored material removal device 350 removes a colored material by using activated carbon or the like. A metal ion removal device 360 removes metal ions by means of a principle such as ion exchange. A buffer tank 370 is provided in the subsequent stage of the foreign matter removal devices 340, 350, 360 in order to temporarily store the fluid mainly composed of BHET or the like after the foreign matter is removed before supplying the fluid to the polymerization reaction tank 400.

A first preheater 371 may be provided in the buffer tank 370 for heating or maintaining the temperature of the depolymerized product (a fluid mainly composed of BHET, or the like) before it is supplied to the subsequent stage polymerization reaction tank 400. The first preheater 371 may maintain the depolymerized product at the same temperature (between 220° C. and 250° C.) as that of the heating unit 320 provided in conjunction with the depolymerization reaction tank 300, or may maintain the depolymerized product at a temperature suitable for a polymerization reaction (between 250° C. and 300° C.), which is the same as that of a heating unit 410 provided in conjunction with the polymerization reaction tank 400 which will be described later. In this way, by providing the buffer tank 370 including a preheating mechanism (first preheater 371) as needed in the preceding stage of the polymerization reaction tank 400, it is possible to store the depolymerized product waiting to be fed to the polymerization reaction tank 400, which typically has a slower processing speed or reaction rate than other processing units such as the depolymerization reaction tank 300 and the by-product removal device 500, which will be described later, while maintaining the depolymerized product at an appropriate temperature. As a result, the capacity of the entire chemical recycling device 100 can be increased, and the chemical recycling device 100 can be stably and continuously operated (without causing so-called “resin shortage”) while an appropriate amount of a reaction product is timely supplied to each of the processing units such as the depolymerization reaction tank 300, the polymerization reaction tank 400, the by-product removal device 500, and the polymer supply unit 600. The preheating mechanism such as the first preheater 371 is not limited to the buffer tank 370, and may be provided at any location (for example, the foreign matter removal devices 340, 350, and 360) between the depolymerization reaction tank 300 and the polymerization reaction tank 400 in any mode.

The polymerization reaction tank 400 synthesizes the depolymerized product such as BHET, which is generated in the depolymerization reaction tank 300 and from which the foreign matter is removed by the foreign matter removal devices 340, 350, and 360, into the polymer through the polymerization reaction. In a case where the depolymerized product generated in the depolymerization reaction tank 300 is BHET, PET, which is the polymer, is obtained again through the polymerization reaction in the polymerization reaction tank 400.

As schematically shown in FIG. 2, EG as a by-product is generated together with PET as a main product, which is a polymer, in the polymerization reaction (400) of BHET as the depolymerized product. The EG may be circulated to the depolymerization material supply unit 310 and used for the depolymerization reaction of PET in the depolymerization reaction tank 300. Since the EG generated in the polymerization reaction tank 400 can be reused on the spot (in the depolymerization reaction tank 300) without being wasted, the operating efficiency of the chemical recycling device 100 can be improved. In particular, the amount of EG to be purchased for the depolymerization reaction of PET in the depolymerization reaction tank 300 can be significantly reduced, so that the operating cost of the chemical recycling device 100 can be reduced.

In order to promote the polymerization reaction as described above, an inside of the polymerization reaction tank 400 is maintained at a suitable temperature for the polymerization reaction by the heating unit 410 (FIG. 1) or the temperature maintaining unit serving as a second heating unit that is provided in conjunction with the polymerization reaction tank 400. The suitable temperature for the polymerization reaction of BHET to PET in FIG. 2 is between 250° C. and 300° C., is preferably between 260° C. and 290° C., and is more preferably between 270° C. and 280° C. Here, a polymerization heating temperature by the heating unit 410 that is provided in conjunction with the polymerization reaction tank 400 is higher than a depolymerization heating temperature by the heating unit 320 that is provided in conjunction with the depolymerization reaction tank 300. Although PET having a large molecular weight and a high melting point is generated in the polymerization reaction tank 400, the temperature of the polymerization reaction tank 400 is maintained higher than a temperature of the depolymerization reaction tank 300 in which BHET having a small molecular weight and a low melting point is generated, so that PET, which is the main product of the polymerization reaction tank 400, is maintained in a molten state. It is preferable that the polymerization reaction of BHET to PET in FIG. 2 is performed in a vacuum state. For this reason, the polymerization reaction tank 400 is provided in conjunction with a vacuum pump or the like (not shown).

The viscosity of the fluid in the polymerization reaction tank 400 in which the PET having a large molecular weight is generated is higher than the viscosity of the fluid in the depolymerization reaction tank 300 in which the BHET having a smaller molecular weight than the PET, which is the polymer, is generated. Therefore, a stirring blade 420 for stirring the fluid in the polymerization reaction tank 400 to promote the polymerization reaction is used for high viscosity. Examples of the stirring blade 420 for high viscosity include an anchor blade and a helical ribbon blade.

As a numerical value correlated with a degree of polymerization of the polymer such as PET, an intrinsic viscosity (IV) value or an inherent viscosity is known. The IV value (dL/g) is also used as an index for the use of the polymer, and in PET, an IV value of about 0.72 or more can be used for a bottle, an IV value of about 0.65 or more can be used for a sheet, a film, or the like, and an IV value of about 0.58 or more can be used for fibers.

In the present embodiment, the object is to finally obtain PET having an IV value that can be used for a bottle or a sheet. As will be described later, the IV value of the PET synthesized in the polymerization reaction tank 400 may be relatively low because the IV value is also increased in the by-product removal device 500 in the subsequent stage of the polymerization reaction tank 400. Specifically, the IV value of the PET synthesized in the polymerization reaction tank 400 is between 0.2 and 0.7, is preferably between 0.3 and 0.7, and is more preferably between 0.3 and 0.55.

A buffer tank 430 that temporarily stores the polymer synthesized in the polymerization reaction tank 400 before supplying the polymer to the by-product removal device 500 in the subsequent stage and/or the polymer supply unit 600 may be provided in the subsequent stage of the polymerization reaction tank 400. A second preheater 431 is provided in the buffer tank 430 for heating or maintaining the temperature of the polymer before it is supplied to the subsequent stage by-product removal device 500 and/or the polymer supply unit 600. The second preheater 431 may maintain the polymer at the same temperature (between 250° C. and 300° C.) as that of the heating unit 410 that is provided in conjunction with the polymerization reaction tank 400, may maintain the polymer at a suitable temperature (between 250° C. and 290° C.) for the polymerization reaction, which is the same as that of a heating unit 520 that is provided in conjunction with the by-product removal device 500 which will be described later, or may maintain the polymer at the same temperature (between 250° C. and 290° C.) as that of a heating unit 620 that is provided in conjunction with the polymer supply unit 600 which will be described later.

In this way, by providing the buffer tank 430 including the preheating mechanism (second preheater 431) as needed in the preceding stage of the by-product removal device 500 and/or the polymer supply unit 600, it is possible to store the polymer waiting to be fed to the by-product removal device 500 and/or the polymer supply unit 600 while maintaining the polymer at an appropriate temperature. As a result, the capacity of the entire chemical recycling device 100 can be increased, and the chemical recycling device 100 can be stably and continuously operated (without causing so-called “resin shortage”) while an appropriate amount of a reaction product is timely supplied to each of the processing units such as the depolymerization reaction tank 300, the polymerization reaction tank 400, the by-product removal device 500, and the polymer supply unit 600. The preheating mechanism such as the second preheater 431 is not limited to the buffer tank 430, and may be provided at any location between the polymerization reaction tank 400 and the by-product removal device 500 and/or at any location between the by-product removal device 500 and the polymer supply unit 600 in any mode.

In the subsequent stage of the polymerization reaction tank 400 (and a preceding stage of the polymer supply unit 600, which will be described later), the by-product removal device 500 is provided through which PET (main product) and EG (by-product) generated through the polymerization reaction in the polymerization reaction tank 400 pass and which removes the EG as the by-product. The by-product removal device 500 in the shown example includes a large number of linear members 510 extending downward from above. Due to the increased surface area provided by the large number of linear members 510, the volatilization of EG adhering to the surface of each linear member 510 is promoted, and the EG is effectively separated and removed from the high-viscosity PET.

The EG may be circulated to the depolymerization material supply unit 310 and used for the depolymerization reaction of PET in the depolymerization reaction tank 300. Since the EG separated and removed in the by-product removal device 500 can be reused on the spot (in the depolymerization reaction tank 300) without being wasted, the operating efficiency of the chemical recycling device 100 can be improved. In particular, the amount of EG to be purchased for the depolymerization reaction of PET in the depolymerization reaction tank 300 can be significantly reduced, so that the operating cost of the chemical recycling device 100 can be reduced.

In addition, the PET having a relatively low degree of polymerization (that is, an IV value) and the BHET which is unreacted in the polymerization reaction tank 400 also adhere to the surface of each of the linear members 510, so that the polymerization reaction similar to that in the polymerization reaction tank 400 effectively progresses due to a large surface area. For this reason, the IV value of the PET as the main product is increased by passing through the by-product removal device 500. Specifically, the IV value of the PET after passing through the by-product removal device 500 is 0.7 or more, is preferably 0.8 or more, and is more preferably 0.85 or more.

In order to promote such a polymerization reaction, an inside of the by-product removal device 500 is maintained at a suitable temperature for the polymerization reaction by the heating unit 520 (FIG. 1) or the temperature maintaining unit serving as the second heating unit that is provided in conjunction with the by-product removal device 500. Specifically, the heating temperature by the heating unit 520 is between 250° C. and 290° C., and preferably between 260° C. and 280° C. Here, the heating temperature by the heating unit 520 that is provided in conjunction with the by-product removal device 500 is preferably higher than a polymerization heating temperature by the heating unit 410 that is provided in conjunction with the polymerization reaction tank 400. In the by-product removal device 500, the polymerization reaction progresses further than in the polymerization reaction tank 400, resulting in a larger molecular weight of PET as a polymer and a higher melting point. By maintaining a higher temperature inside the by-product removal device 500 than inside the polymerization reaction tank 400, the PET as a product of the by-product removal device 500 can be maintained in a molten state. A heating unit or a temperature maintaining unit, serving as a second heating unit, for heating or maintaining the temperature at least at the polymerization heating temperature by the heating unit 410 that is provided in conjunction with the polymerization reaction tank 400 may be provided around a pipe or the like between the polymerization reaction tank 400 and the by-product removal device 500. Further, it is preferable that the polymerization reaction in the by-product removal device 500 is performed in a vacuum state in the same manner as the polymerization reaction in the polymerization reaction tank 400. For this reason, the by-product removal device 500 is provided in conjunction with a vacuum pump or the like (not shown). The EG as the by-product can be efficiently removed by setting the inside of the by-product removal device 500 in a vacuum state (reduced pressure state).

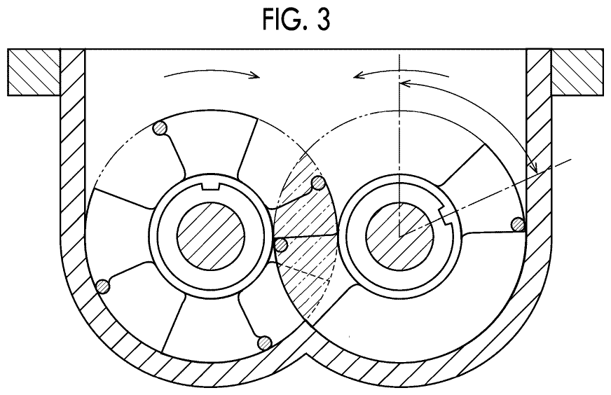

The configuration of the by-product removal device 500 is not limited to a “vertical type” as shown in FIG. 1. For example, a stirring device of a “two-shaft horizontal type” as shown in FIG. 3 may be used as the by-product removal device 500. The stirring device includes two rotary shafts extending in a direction perpendicular to the paper surface of FIG. 3, and two stirring blades that rotate around each of the rotary shafts to stir PET and EG as stirring targets. The volatilization of the EG is promoted by being stirred by the two stirring blades, so that the EG is effectively separated and removed from the high-viscosity PET. The details of the stirring device in FIG. 3 are disclosed in Japanese Patent Application No. 2925599, which is incorporated herein by reference.

The polymer supply unit 600 supplies the polymer such as PET synthesized in the polymerization reaction tank 400 (or the polymerization reaction tank 400 and the by-product removal device 500) to the injection molding machine 1 that molds the second molding product such as a PET bottle. The polymer supply unit 600 includes a transfer pump 610 such as a gear pump or a screw pump suitable for supplying the high-purity and high-viscosity (that is, high degree of polymerization or high IV value) PET from which the EG as the by-product is removed in by-product removal device 500 to the injection molding machine 1 in a molten state.

The polymer supply unit 600 is provided with the heating unit 620 or the temperature maintaining unit as a first heating unit for heating or maintaining the temperature of the polymer such as PET to be transferred to the injection molding machine 1 by the transfer pump 610 to maintain it in a molten state. Specifically, the heating temperature by the heating unit 620 is between 250° C. and 290° C., and preferably between 260° C. and 280° C. Here, the heating temperature (first heating temperature) by the heating unit 620 (first heating unit) provided in the polymer supply unit 600 is preferably higher than a second heating temperature by the second heating unit such as the heating unit 410 provided in conjunction with the polymerization reaction tank 400, the heating unit 520 provided in conjunction with the by-product removal device 500, and a heating unit (not shown) provided between the polymerization reaction tank 400 and the by-product removal device 500. The polymerization reaction that begins in the polymerization reaction tank 400 gradually progresses and is completed in the by-product removal device 500. As a result, the molecular weight of the polymer such as PET in the polymer supply unit 600 becomes larger and the melting point thereof becomes higher than in the polymerization reaction tank 400 and the by-product removal device 500. Therefore, by making the first heating temperature in the polymer supply unit 600 higher than the previous second heating temperature, the polymer such as PET having a high viscosity (that is, high degree of polymerization or high IV value) and a high melting point can be maintained in a molten state. In addition, the polymer supply unit 600 may be provided in conjunction with a vacuum pump (not shown) or the like. The degree of polymerization can also be increased in the polymer supply unit 600 by setting the inside of the polymer supply unit 600 in a vacuum state (reduced pressure state).

A temperature gradient may be provided such that the heating temperature increases stepwise from the polymerization reaction tank 400 to the polymer supply unit 600. For example, by making the heating temperature by a heating unit (not shown) provided between the polymerization reaction tank 400 and the by-product removal device 500 higher than the heating temperature by the heating unit 410 provided in conjunction with the polymerization reaction tank 400, making the heating temperature by the heating unit 520 provided in conjunction with the by-product removal device 500 higher than the heating temperature by the heating unit (not shown), and making the heating temperature by the heating unit 620 provided in the polymer supply unit 600 higher than the heating temperature by the heating unit 520, it is possible to reliably maintain the polymer such as PET, whose melting point increases from the polymerization reaction tank 400 to the polymer supply unit 600, in a molten state. A heating unit for heating or maintaining the temperature of the polymer such as PET to maintain it in a molten state may be provided between the polymer supply unit 600 and the injection molding machine 1.

The injection molding machine 1 molds the polymer, such as PET, in a molten state generated by the chemical recycling device 100 into a second molding product. The second molding product may be the same as or different from the first molding product that is subjected to a pulverizing process or the like by the polymer adjustment device 200. For example, the first molding product and the second molding product may both be PET bottles. In addition, one of the first molding product and the second molding product may be a PET bottle, and the other may be a molding product other than a bottle, such as a sheet, a film, and a fiber. In general, in mechanical recycling, the IV value of the second molding product after recycling becomes lower than the IV value of the first molding product before recycling. However, according to the chemical recycling device 100 of the present embodiment including the mechanism for increasing the IV values such as the foreign matter removal devices 340, 350, and 360 and the by-product removal device 500, it is also possible to increase the IV value of the second molding product after recycling to be higher than the IV value of the first molding product before recycling. For example, according to the present embodiment, the PET fiber having a low IV value as the first molding product can be recycled into the PET bottle having a high IV value as the second molding product.

The injection molding machine 1 molds a molten resin such as PET into a second molding product. An injection molding machine that uses a molten resin as a raw material is disclosed in the related art. The present application incorporates by reference the entire contents of the literature (Japanese Patent Application No. 2020-130985) filed on Jul. 31, 2020. As schematically shown in FIG. 1, a plurality of the injection molding machines 1 may be provided in parallel. The molding machine to which the molten resin or the like is supplied from the chemical recycling device 100 is not limited to an injection molding machine, and may be any molding machine (for example, a compression molding machine).

In the present embodiment as described above, the polymer resynthesized in the polymerization reaction tank 400 is not made into flakes or pellets, and is supplied as it is to the injection molding machine 1 by the polymer supply unit 600. Since the cooling process and the heating process related to the flakes or the pellets as in the related art are not required, the molding product such as a PET bottle can be recycled with less energy as compared with the related art.

In the chemical recycling device 100 according to the present embodiment, since the polymer resynthesized in the polymerization reaction tank 400 is supplied as it is to the injection molding machine 1, it is necessary to quickly realize the IV value of the polymer required for the molding product (second molding product). In the present embodiment, the by-product removal device 500 having a function of promoting the polymerization reaction and increasing the IV value of the polymer is provided in addition to the polymerization reaction tank 400. Therefore, the by-product removal device 500 can sufficiently meet such a requirement.

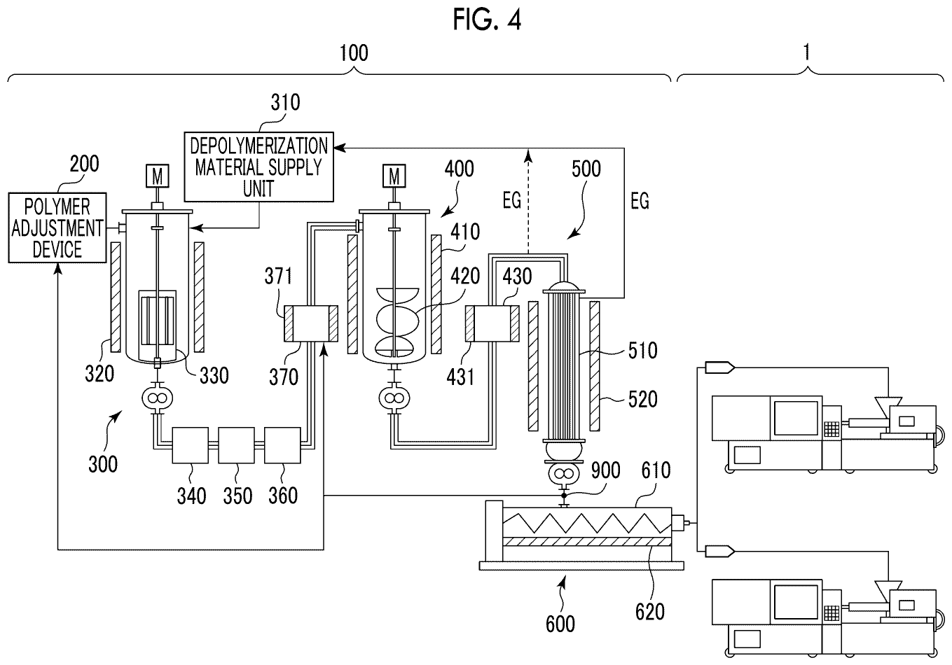

FIG. 4 schematically shows a configuration of a chemical recycling molding system according to another embodiment of the present invention. The same components as those in FIG. 1 are denoted by the same reference numerals, and overlapping description is omitted. In the present embodiment, a polymer return unit 900 that returns at least a part of the polymer synthesized in the polymerization reaction tank 400 to the polymerization reaction tank 400 and/or the depolymerization reaction tank 300 is provided. The polymer return unit 900 in the shown example is provided between the by-product removal device 500 and the polymer supply unit 600, but is not limited to this. The polymer return unit 900 may be provided at any position between the by-product removal device 500 and the injection molding machine 1. The polymer return unit 900 may be provided, for example, between the polymer supply unit 600 and the injection molding machine 1.

The polymer return unit 900 returns at least a part of the polymer (in the shown example, the polymer from which the by-product is further removed by the by-product removal device 500) synthesized in the polymerization reaction tank 400 to the polymerization reaction tank 400 and/or the depolymerization reaction tank 300 instead of sending it to the polymer supply unit 600. For example, the polymer return unit 900 may supply the polymer to the buffer tank 370 provided in the preceding stage of the polymerization reaction tank 400 in order to return the polymer to the polymerization reaction tank 400, or may supply the polymer to the polymer adjustment device 200 provided in the preceding stage of the depolymerization reaction tank 300 in order to return the polymer to the depolymerization reaction tank 300.

The main purpose of the polymer return unit 900 is to cope with a mismatch in the operation time and the capacity between the chemical recycling device 100 and the injection molding machine 1. For example, the chemical recycling device 100 typically operates continuously (continuous operation), and the injection molding machine 1 typically operates intermittently (intermittent operation). Therefore, particularly while the injection molding machine 1 is stopped for maintenance of a mold or the like, the injection molding machine 1 cannot accept the total amount of the polymer from the polymer supply unit 600 (unacceptable period). Even during such an unacceptable period, the surplus polymer that cannot be accepted by the injection molding machine 1 is returned to the polymerization reaction tank 400 and/or the depolymerization reaction tank 300 in a molten state so that the chemical recycling device 100 can be continuously operated without wasting the surplus polymer. In this way, since it is no longer necessary to make the surplus polymer in a molten state that cannot be accepted by the injection molding machine 1 into flakes or pellets by cooling or releasing heat, the waste of energy is reduced.

The polymer return unit 900 may change a return destination (polymerization reaction tank 400 and/or depolymerization reaction tank 300) or a return ratio of the surplus polymer depending on the length of the unacceptable period. For example, before the unacceptable period exceeds a predetermined period threshold, more of the polymer may be returned to the polymerization reaction tank 400 than to the depolymerization reaction tank 300, and after the unacceptable period exceeds the period threshold, more of the polymer may be returned to the depolymerization reaction tank 300 than to the polymerization reaction tank 400.

In a case where the unacceptable period is a short period which does not exceed the period threshold, it is considered that the deterioration of the polymer or the excessive increase in the IV value does not occur even though the polymer in a molten state is circulated through the polymerization reaction tank 400 (and the by-product removal device 500) at a high temperature (for example, between 270° C. and 280° C. as described above). Therefore, it is preferable that the polymer return unit 900 returns most (for example, 100%, 80%, 60%) of the surplus polymer to the polymerization reaction tank 400 (buffer tank 370) for the short unacceptable period.

On the other hand, in a case where the unacceptable period exceeds the period threshold and becomes a long period, there is a risk that the polymer may deteriorate or that the IV value may excessively increase due to the heat history caused by the circulation of the polymer in a molten state in the polymerization reaction tank 400 (and the by-product removal device 500) at a high temperature (for example, between 270° C. and 280° C. as described above). Therefore, it is preferable that the polymer return unit 900 returns most (for example, 100%, 80%, 60%) of the surplus polymer to the depolymerization reaction tank 300 (polymer adjustment device 200) for the long unacceptable period. Since the above-described heat history is substantially reset by another depolymerization reaction in the depolymerization reaction tank 300, the deterioration of the polymer or the excessive increase in the IV value of the polymer supplied to the injection molding machine 1 by the polymer supply unit 600 after the unacceptable period are effectively prevented.

In the present embodiment, since the temperature of the circulating polymer in a molten state is substantially constant (for example, between 270° C. and 280° C. as described above), a length of the unacceptable period represents the heat history. On the other hand, in a case where the temperature of the circulating polymer in a molten state can significantly change, the heat history may be strictly identified by monitoring the temperature in addition to the length of the unacceptable period. In this case, the polymer return unit 900 may return more of the polymer to the polymerization reaction tank 400 than to the depolymerization reaction tank 300 before the heat history exceeds a predetermined heat history threshold, and return more of the polymer to the depolymerization reaction tank 300 than to the polymerization reaction tank 400 after the heat history exceeds the heat history threshold.

The polymer return unit 900 may change the return destination (polymerization reaction tank 400 and/or depolymerization reaction tank 300) or the return ratio depending on physical property values such as an IV value of the polymer to be returned. For example, in a case where the IV value is within a predetermined allowable range, more of the polymer may be returned to the polymerization reaction tank 400 than to the depolymerization reaction tank 300, and in a case where the IV value is out of the allowable range (specifically, in a case where the IV value is larger than the upper limit of the allowable range), more of the polymer may be returned to the polymerization reaction tank 400 than to the depolymerization reaction tank 300. In the latter case, the IV value excessively increased is reduced to be within the allowable range through another depolymerization reaction in the depolymerization reaction tank 300.

In addition, the polymer return unit 900 may gradually change the return ratio to the polymerization reaction tank 400 and the depolymerization reaction tank 300 depending on the length of the unacceptable period and the physical property values such as the IV value of the polymer to be returned. For example, the return ratio to the depolymerization reaction tank 300 may be gradually increased as the unacceptable period becomes longer, or the return ratio to the depolymerization reaction tank 300 may be gradually increased as the IV value increases.

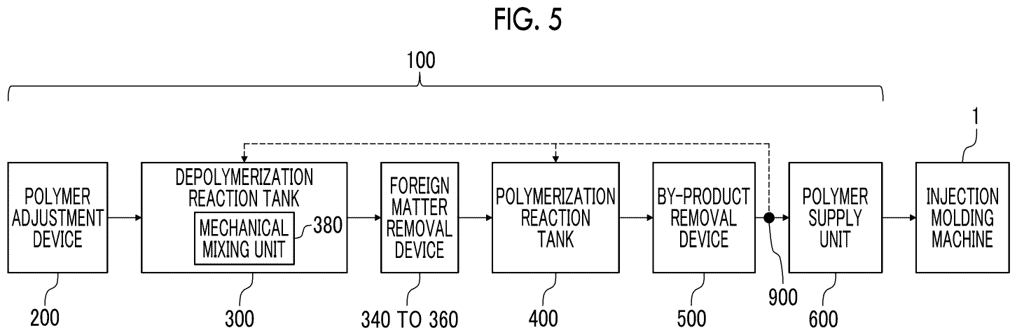

FIG. 5 schematically shows a configuration of a chemical recycling molding system according to still another embodiment of the present invention. The same components as those in the above-described embodiment are denoted by the same reference numerals, and overlapping description is omitted. In addition, in FIG. 5 and subsequent similar drawings, the main components of the chemical recycling molding system are shown as functional blocks for convenience. In the present embodiment, a mechanical mixing unit 380 that mechanically or physically mixes the depolymerization material and the polymer is provided in the depolymerization reaction tank 300 at least in an early stage of the depolymerization reaction of decomposing the polymer into the depolymerized product by using the depolymerization material.

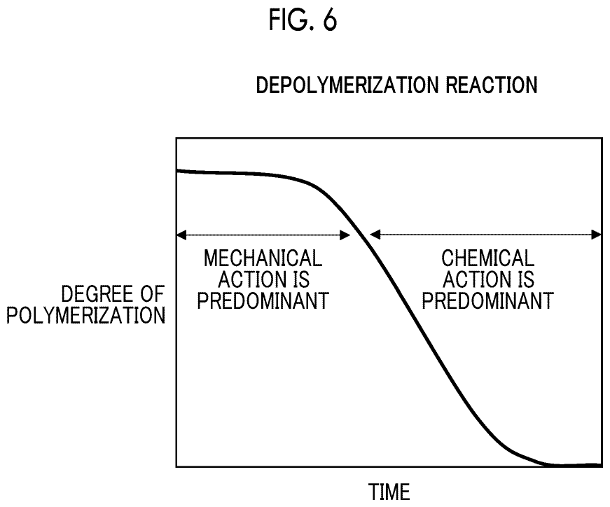

As shown in FIG. 2, the depolymerization reaction of decomposing the polymer such as PET into the depolymerized product such as BHET by using the depolymerization material such as EG is essentially a chemical process. However, as schematically shown in FIG. 6, in an initial stage or the early stage of the depolymerization reaction when the degree of polymerization of the polymer is high, the polymer such as PET exists in a large lump such as flakes or pellets, and a mechanical action such as stirring or mixing is predominant over a chemical action (chemical reaction). On the other hand, in a latter stage of the depolymerization reaction, the desired chemical action (chemical reaction) becomes predominant.

In particular, the mechanical mixing unit 380 according to the present embodiment mechanically mixes the depolymerization material such as EG and the polymer such as PET in the initial stage or the early stage of the depolymerization reaction in which the mechanical action is predominant to efficiently advance the period and enable a quick transition to the latter stage in which the desired chemical action (chemical reaction) is predominant. As a result, the depolymerization reaction in the depolymerization reaction tank 300 can proceed efficiently in a short period of time. The mechanical mixing unit 380 may continue the mechanical mixing of the depolymerization material such as EG and the polymer such as PET not only in the initial stage or the early stage of the depolymerization reaction in which the mechanical action is predominant but also in the latter stage of the depolymerization reaction in which the chemical action (chemical reaction) is predominant. The mechanical mixing unit 380 can perform the processes such as pulverizing, heating and melting, and mixing, as in the above-described polymer adjustment device 200. Therefore, the polymer adjustment device 200 may not be provided, or the polymer adjustment device 200 may be integrated with the mechanical mixing unit 380.

The mechanical mixing unit 380 as described above may be provided in conjunction with or external to a single-tank configuration depolymerization reaction tank 300 as shown in FIGS. 1 and 4. For example, an ultrasonic device, which will be described later, as the mechanical mixing unit 380 may be attached to an outer wall surface and/or an inner wall surface of the depolymerization reaction tank 300, or a cavitation device, which will be described later, as the mechanical mixing unit 380 may be provided in a preceding stage of the depolymerization reaction tank 300 (for example, on a supply path on which the depolymerization material supply unit 310 supplies the depolymerization material such as EG to the depolymerization reaction tank 300).

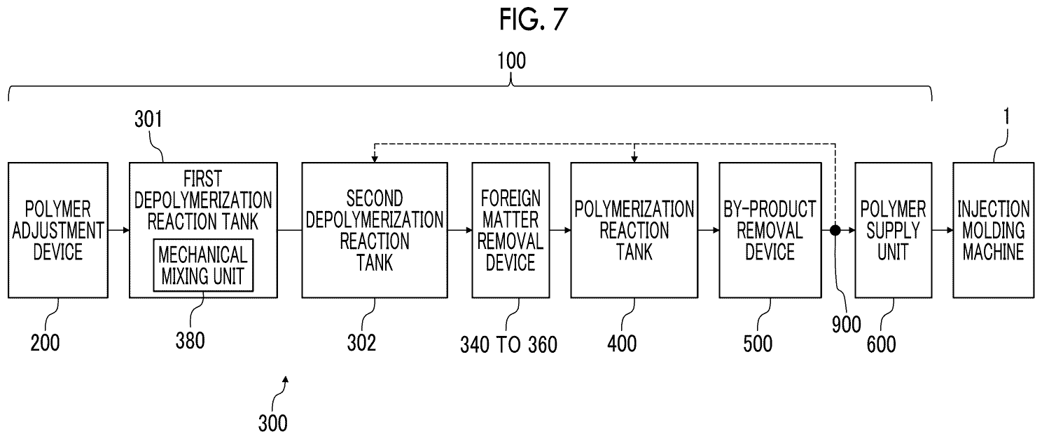

Alternatively, as schematically shown in FIG. 7, the depolymerization reaction tank 300 may include a plurality of tanks (or two tanks) including a first depolymerization reaction tank 301 in the preceding stage and a second depolymerization reaction tank 302 in the subsequent stage, and the mechanical mixing unit 380 may be provided in at least the first depolymerization reaction tank 301. The first depolymerization reaction tank 301 in the preceding stage is mainly responsible for the initial stage or the early stage of the depolymerization reaction schematically shown in FIG. 6, in which the mechanical action is predominant. By providing the mechanical mixing unit 380 in the first depolymerization reaction tank 301, the depolymerization material such as EG and the polymer such as PET are mechanically mixed with each other in the initial stage or the early stage of the depolymerization reaction in which the mechanical action is predominant, and thereby the period can be efficiently advanced. The second depolymerization reaction tank 302 in the subsequent stage is mainly responsible for the latter stage of the depolymerization reaction schematically shown in FIG. 6, in which the chemical action (chemical reaction) is predominant. The second depolymerization reaction tank 302 may be configured in the same manner as the depolymerization reaction tank 300 as shown in FIGS. 1 and 4.

The mechanical mixing unit 380 as described above may be configured in any manner as long as the mechanical mixing unit 380 has a function of mechanically mixing the depolymerization material such as EG and the polymer such as PET, particularly in the initial stage of the depolymerization reaction. Hereinafter, a plurality of non-limiting examples of the mechanical mixing unit 380 provided in the first depolymerization reaction tank 301 in FIG. 7 will be described.

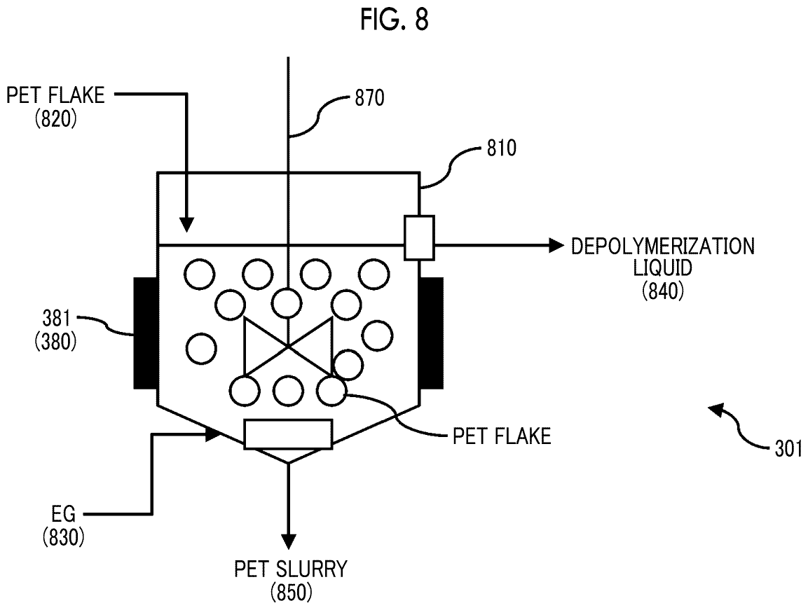

FIG. 8 schematically shows a first example of the mechanical mixing unit 380. In the present example, the first depolymerization reaction tank 301 is configured by a mixing tank 810, a polymer supply unit 820, a depolymerization material supply unit 830, a depolymerized product discharge unit 840, a polymer take-out unit 850, a stirring blade 870, and an ultrasonic device 381 as the first example of the mechanical mixing unit 380.

In the mixing tank 810, a partial depolymerization reaction of a polymer such as PET flakes, particularly, mechanical mixing with a depolymerization material such as EG in the initial stage or the early stage described above with reference to FIG. 6, is performed. The polymer supply unit 820 supplies the polymer such as PET flakes into the mixing tank 810. The depolymerization material supply unit 830 provided at a bottom portion of the mixing tank 810 supplies the depolymerization material such as EG for decomposing the polymer such as PET to the mixing tank 810. The depolymerization material supply unit 830 may supply a washing liquid for washing a PET slurry or the like remaining after the depolymerization reaction (in particular, mechanical mixing) to the mixing tank 810. The depolymerized product discharge unit 840 provided at an upper portion of the mixing tank 810 discharges a liquid-state depolymerized product such as BHET generated through the depolymerization reaction. Although not shown, the depolymerized product such as BHET discharged from the depolymerized product discharge unit 840 may be supplied to the foreign matter removal devices 340 to 360 together with the depolymerized product such as BHET discharged from the second depolymerization reaction tank 302. In addition, the depolymerized product discharge unit 840 is not limited to the depolymerized product such as BHET, and can discharge any liquid contained in the mixing tank 810, and may discharge, for example, the above-described washing liquid. The polymer take-out unit 850 takes out the PET slurry or the like remaining after the depolymerization reaction. The PET slurry or the like taken out from the polymer take-out unit 850 is sent to the second depolymerization reaction tank 302 in the subsequent stage, and is decomposed into the depolymerized product such as BHET through a further depolymerization reaction (in particular, a desired chemical reaction in the latter stage described above with reference to FIG. 6).

The stirring blade 870 and/or the ultrasonic device 381 mechanically mix or stir the polymer such as PET supplied from the polymer supply unit 820 and the depolymerization material such as EG supplied from the depolymerization material supply unit 830 in the mixing tank 810 to promote mechanical mixing in the initial stage or the early stage of the depolymerization reaction which is performed by the first depolymerization reaction tank 301. Through such mechanical mixing, the depolymerization reaction efficiently progresses, and the PET flakes or the like are also finely divided to form a slurry state. The finely divided PET slurry can pass through the polymer take-out unit 850 constituted by a strainer and a valve, and is taken out to the outside of the mixing tank 810, and is sent to the second depolymerization reaction tank 302.

The ultrasonic device 381 is attached, for example, continuously (for example, in an annular shape) or intermittently at any location on the outer wall surface and/or the inner wall surface of the mixing tank 810. Ultrasonic waves (vibration) generated by the ultrasonic device 381 mechanically mix the polymer such as PET and the depolymerization material such as EG in the mixing tank 810. In a case where it is difficult to attach the ultrasonic device 381 to the wall surface of the mixing tank 810, another ultrasonic tank in which the ultrasonic device 381 is incorporated may be connected to the mixing tank 810. In this case, the mechanical mixing by the ultrasonic device 381 is performed in the ultrasonic tank, and the PET or the like that is finely divided is taken out from the polymer take-out unit 850 of the mixing tank 810.

For example, the configuration of FIG. 8 is realized by the following procedure to realize the desired function (mainly, mechanical mixing) of the first depolymerization reaction tank 301. First, in a first step, the polymer supply unit 820 supplies the polymer such as PET flakes to the mixing tank 810, and the depolymerization material supply unit 830 supplies the depolymerization material such as EG to the mixing tank 810. In this case, the inside of the mixing tank 810 is maintained at the same temperature (for example, between 220° C. and 250° C., preferably between 230° C. and 245° C., and more preferably between 235° C. and 240° C.) and the same pressure (for example, between 0.3 MPa and 0.8 MPa, preferably between 0.4 MPa and 0.6 MPa, and more preferably between 0.45 MPa and 0.55 MPa) as those in the depolymerization reaction tank 300 in FIG. 1. Therefore, in the mixing tank 810, an initial depolymerization reaction (mechanical action in FIG. 6) substantially the same as that generated in the depolymerization reaction tank 300 in FIG. 1 is generated. However, according to the configuration of FIG. 8, the mechanical mixing is particularly promoted by the ultrasonic device 381 as described above. In a case where the PET flakes or the like are too large, it is difficult to stir them with the contact type stirring blade 870. However, the PET flakes or the like can be effectively mixed regardless of the size of the PET flakes or the like with the non-contact type ultrasonic device 381.

After the mechanical mixing of the polymer such as the PET flakes sufficiently progresses in the mixing tank 810 (that is, after the first step), or while the mechanical mixing progresses (that is, at the same time as the first step), a depolymerization liquid containing the depolymerized product such as BHET generated through the depolymerization reaction is discharged from the depolymerized product discharge unit 840 provided in the upper portion of the mixing tank 810, as a second step. The depolymerized product discharge unit 840 includes a strainer and a valve capable of discharging the liquid at all times or intermittently. The liquid-state depolymerization material such as EG supplied from the depolymerization material supply unit 830 or a catalyst and/or the liquid-state depolymerized product such as BHET generated through the depolymerization reaction fills the mixing tank 810 from the bottom portion to a position where the depolymerized product discharge unit 840 is provided. In this way, a liquid level in the mixing tank 810 is formed at the position where the depolymerized product discharge unit 840 is provided. The excessive liquid is discharged from the depolymerized product discharge unit 840 including a strainer and a valve. A plurality of the depolymerized product discharge units 840 may be provided at different heights. In this case, the depolymerized product discharge unit 840 provided at the highest position has a function of preventing overflow and forming a liquid level in the mixing tank 810, and the depolymerized product discharge unit 840 provided at a position lower than the highest position discharges the depolymerized product such as BHET from an intermediate height position.

After the mechanical mixing of the polymer such as the PET flakes and the depolymerization material such as EG sufficiently progresses in the mixing tank 810, as a third step after the first step, the PET slurry, which is finely divided and deposited, is taken out from the polymer take-out unit 850 provided at the bottom portion of the mixing tank 810.

The first to third steps as described above may be continuously executed (continuous processing) by continuing the supply of the polymer such as the PET flakes by the polymer supply unit 820 and the supply of the depolymerization material such as EG by the depolymerization material supply unit 830, or may be executed for each predetermined processing unit or batch of the polymer such as the PET flakes and the depolymerization material such as EG (batch processing). For example, in the continuous processing, while the ultrasonic device 381 continuously emits ultrasonic waves and/or the stirring blade 870 continuously rotates in the mixing tank 810, at least one of the following is executed continuously or intermittently: supply of the polymer (such as PET flakes) from the polymer supply unit 820, supply of the depolymerization material from the depolymerization material supply unit 830, discharge of the depolymerized product from the depolymerized product discharge unit 840, and taking out of the polymer (such as PET slurry) from the polymer take-out unit 850.

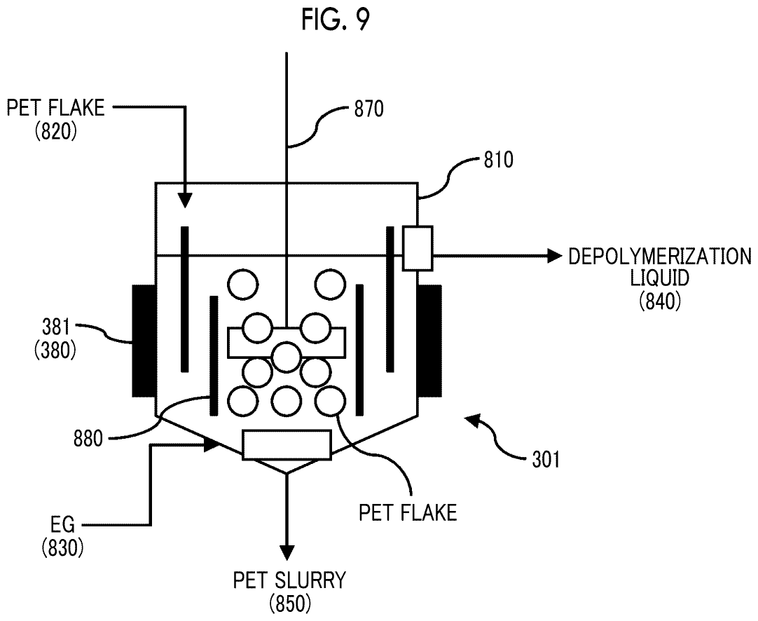

In the modification example in FIG. 9, the first depolymerization reaction tank 301 is configured by the mixing tank 810, the polymer supply unit 820, the depolymerization material supply unit 830, the depolymerized product discharge unit 840, the polymer take-out unit 850, the stirring blade 870, a baffle 880, and the ultrasonic device 381 as the first example of the mechanical mixing unit 380.

In this modification example, in a case where the polymer such as the PET flakes is mechanically mixed by the stirring blade 870 and/or the ultrasonic device 381, the polymer is efficiently and finely divided to form a slurry state by colliding with the baffle 880, which is also called a baffle plate.

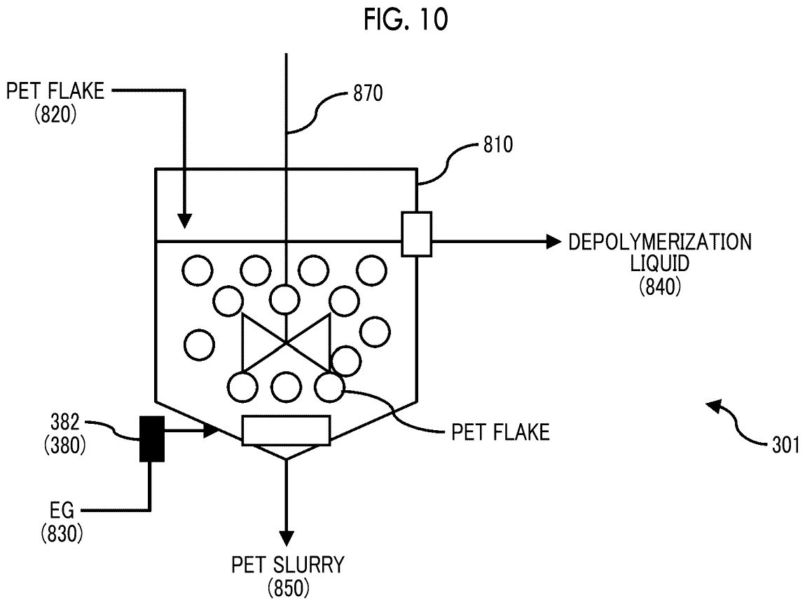

FIG. 10 schematically shows a second example of the mechanical mixing unit 380. In the present example, the first depolymerization reaction tank 301 is configured by the mixing tank 810, the polymer supply unit 820, the depolymerization material supply unit 830, the depolymerized product discharge unit 840, the polymer take-out unit 850, the stirring blade 870, and a cavitation device 382 as the second example of the mechanical mixing unit 380. The same components as those in FIG. 8 are denoted by the same reference numerals, and overlapping description is omitted.

The stirring blade 870 and/or the cavitation device 382 mechanically mix or stir the polymer such as PET supplied from the polymer supply unit 820 and the depolymerization material such as EG supplied from the depolymerization material supply unit 830 in the mixing tank 810 to promote mechanical mixing in the initial stage or the early stage of the depolymerization reaction which is performed by the first depolymerization reaction tank 301. Through such mechanical mixing, the depolymerization reaction efficiently progresses, and the PET flakes or the like are also finely divided to form a slurry state. The finely divided PET slurry can pass through the polymer take-out unit 850 constituted by a strainer and a valve, and is taken out to the outside of the mixing tank 810, and is sent to the second depolymerization reaction tank 302.

For example, the cavitation device 382 is provided on a supply path on which the depolymerization material supply unit 830 supplies the depolymerization material such as EG to the mixing tank 810. The cavitation device 382 introduces a pressure difference into the liquid-state depolymerization material such as EG to cause cavitation (cavity phenomenon) accompanied by generation and collapse of bubbles. Typically, the minute bubbles associated with the cavitation mechanically mix the polymer such as PET and the depolymerization material such as EG in the mixing tank 810. In a case where it is difficult to provide the cavitation device 382 in the first depolymerization reaction tank 301, another cavitation tank in which the cavitation device 382 is incorporated may be connected to the mixing tank 810. In this case, the mechanical mixing by the cavitation device 382 is performed in the cavitation tank, and the PET or the like that is finely divided is taken out from the polymer take-out unit 850 of the mixing tank 810. In addition, instead of or in addition to the cavitation device 382, a bubble generating device that generates minute bubbles (also referred to as fine bubbles or microbubbles) using a principle different from cavitation may be used as the mechanical mixing unit 380.

According to the configuration of FIG. 10, in the above-described first step for realizing the desired function (mainly, mechanical mixing) of the first depolymerization reaction tank 301, the cavitation device 382 generates minute bubbles associated with the cavitation in the depolymerization material such as EG supplied to the mixing tank 810 by the depolymerization material supply unit 830. These minute bubbles are introduced into the mixing tank 810 together with the depolymerization material such as EG, and mechanically mix the polymer such as the PET flakes supplied by the polymer supply unit 820 and the depolymerization material such as EG supplied by the depolymerization material supply unit 830. In the mixing tank 810, the initial depolymerization reaction (mechanical action in FIG. 6) substantially the same as that generated in the depolymerization reaction tank 300 in FIG. 1 is generated. However, according to the configuration of FIG. 10, the mechanical mixing is particularly promoted by the cavitation device 382 as described above. In a case where the PET flakes or the like are too large, it is difficult to stir them with the contact type stirring blade 870. However, the PET flakes or the like can be effectively mixed regardless of the size of the PET flakes or the like with the non-contact type cavitation device 382.

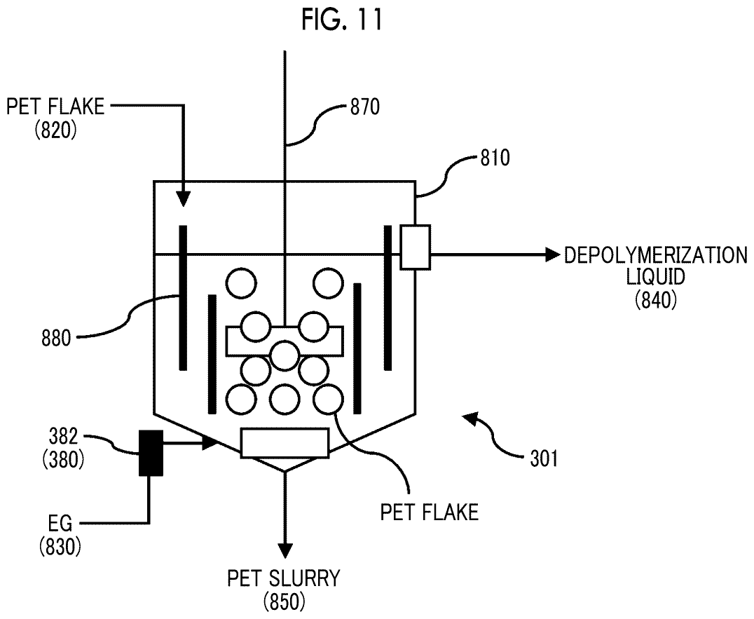

In the modification example in FIG. 11, the first depolymerization reaction tank 301 is configured by the mixing tank 810, the polymer supply unit 820, the depolymerization material supply unit 830, the depolymerized product discharge unit 840, the polymer take-out unit 850, the stirring blade 870, the baffle 880, and the cavitation device 382 as the second example of the mechanical mixing unit 380.

In this modification example, in a case where the polymer such as the PET flakes is mechanically mixed by the stirring blade 870 and/or the cavitation device 382, the polymer is efficiently and finely divided to form a slurry state by colliding with the baffle 880, which is also called a baffle plate.

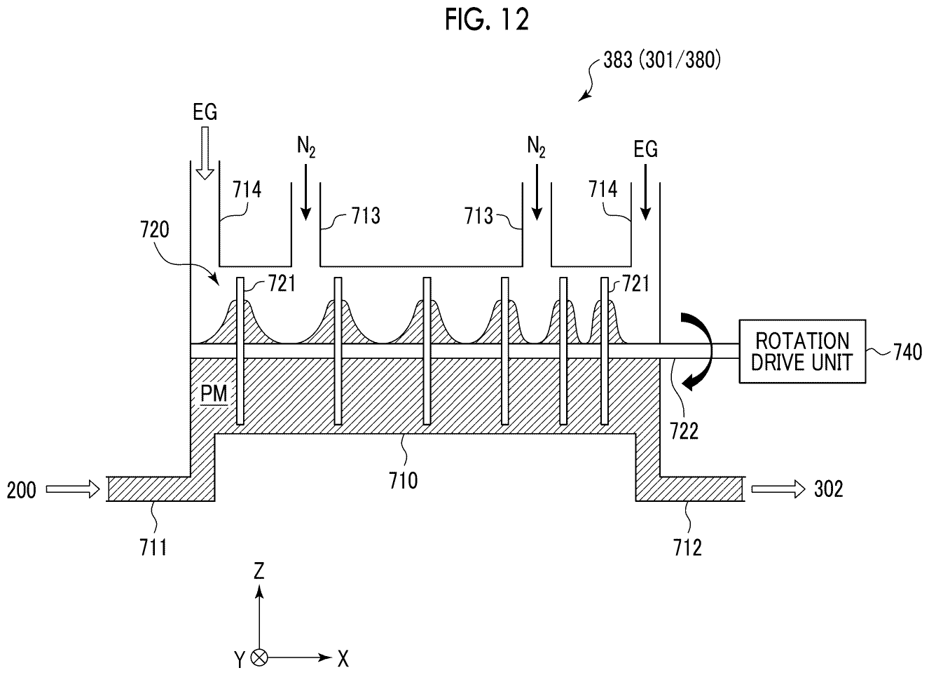

FIG. 12 schematically shows a horizontal type stirring device 383 as a third example of the mechanical mixing unit 380. The horizontal type stirring device 383 constitutes the first depolymerization reaction tank 301 in FIG. 7. In addition, the second depolymerization reaction tank 302 in FIG. 7 is a vertical type stirring device such as the depolymerization reaction tank 300 shown in FIGS. 1 and 4. Here, the term “horizontal type” means that the stirring is performed by using a rotating body rotating around a rotary shaft in a direction intersecting a vertical direction (for example, a horizontal direction), and the term “vertical type” means that the stirring is performed by using a rotating body (for example, a stirring blade) rotating around a rotary shaft in a direction intersecting the horizontal direction (for example, the vertical direction).

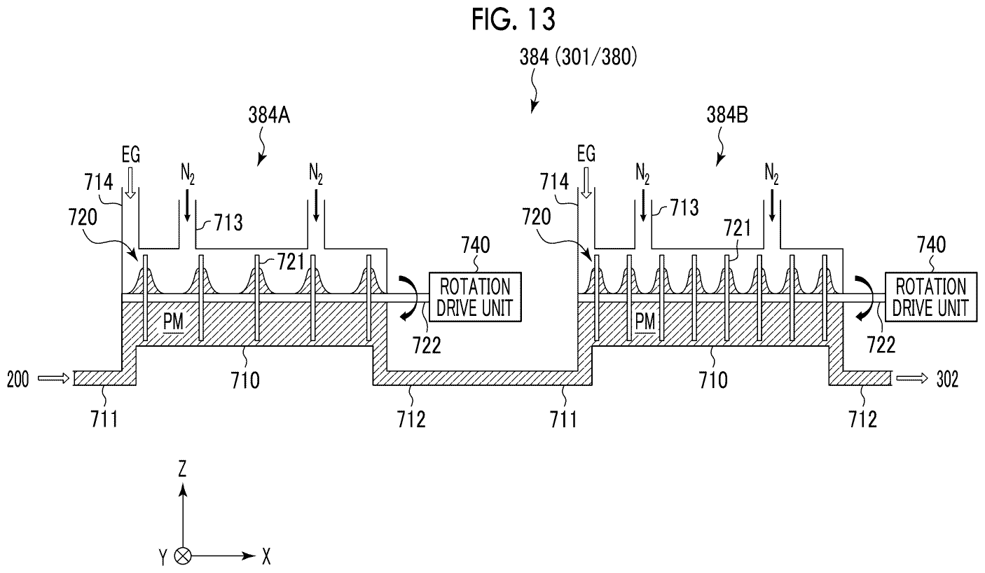

In FIG. 12 (and FIG. 13 which will be described later), a three-dimensional coordinate system is set with an X axis, a Y axis, and a Z axis for convenience. For example, the X axis and the Y axis are in the horizontal direction, and the Z axis is in the vertical direction. The horizontal type stirring device 383 in the shown example is long in an X direction.

The horizontal type stirring device 383 includes a container 710, a rotating body 720, and a rotation drive unit 740.

For example, a polymer PM such as PET derived from the first molding product is supplied to the container 710 from the polymer adjustment device 200 in FIG. 7 in a molten state. The container 710 includes a polymer feed port 711 that is connected to the polymer adjustment device 200 and that supplies the polymer PM into the container 710, and a discharge port 712 that discharges the polymer PM (including the unreacted depolymerization material and the depolymerized product such as BHET as the product) that is stirred (mechanically mixed) with the depolymerization material such as EG by the rotating body 720, which will be described later, outside the container 710, and supplies the polymer PM to the second depolymerization reaction tank 302. A transfer pump such as a gear pump or a screw pump capable of adjusting a supply amount or a supply speed of the polymer PM to the inside of the container 710 may be connected to the polymer feed port 711, and a transfer pump such as a gear pump or a screw pump capable of adjusting a discharge amount or a discharge speed of the polymer PM to the outside of the container 710 may be connected to the discharge port 712. In addition, the container 710 may be provided with a temperature control unit such as a heater that controls the temperature of the inside of the container 710 to an appropriate temperature so that the polymer PM is maintained in a molten state.

The container 710 is long in the X direction as shown in FIG. 12, and has, for example, a substantially rectangular YZ cross section (not shown). In a case where a plurality of the rotating bodies 720 are disposed to be shifted in a Y direction, the YZ cross section of the container 710 is long in the Y direction (that is, a rectangle) to accommodate the plurality of rotating bodies 720.

In FIG. 12, the polymer feed port 711 is provided at one end portion (left end portion in FIG. 12) of the container 710 in the X direction, and the discharge port 712 is provided at the other end portion (right end portion in FIG. 12) of the container 710 in the X direction. In an X-direction region between the polymer feed port 711 and the discharge port 712, one or a plurality of air feed ports 713 for sending a gas such as nitrogen to a gas phase in the container 710 and one or a plurality of depolymerization material feed ports 714 for supplying the depolymerization material such as EG that is mechanically mixed with the polymer PM to the gas phase in the container 710 are provided. In addition, one or a plurality of rotating bodies 720 are provided in the X-direction region between the polymer feed port 711 and the discharge port 712 to stir (mechanically mix) the polymer PM in a molten state supplied from the polymer feed port 711 and the depolymerization material such as EG supplied from the depolymerization material feed port 714.

The rotating body 720 rotates in the container 710 and stirs the polymer PM and the depolymerization material. The rotating body 720 includes one or a plurality of rotating plates 721 rotating around a rotary shaft 722 in a direction (in the example of FIG. 12, the X direction orthogonal to a Z direction) intersecting the vertical direction (in the example of FIG. 12, the Z direction). The rotating plate 721 is, for example, a circular plate having a circular YZ cross section. However, the shape of the YZ cross section of the rotating plate 721 is arbitrary, and may be, for example, an ellipse or a polygon such as a triangle and a quadrangle. It is preferable that the center or the center of gravity of the rotating plate 721 in a YZ plane coincides with the center of the rotary shaft 722. In this case, one or the plurality of rotating plates 721 and the rotary shaft 722 are coaxially disposed. The rotating body 720 may include a screw instead of or in addition to the rotating plate 721. In a case where the rotating body 720 includes the screw, the polymer PM such as PET may be supplied in a solid state.

In the example of FIG. 12, the rotating body 720 includes a plurality of rotating plates 721 separated apart in an axial direction (X direction) of the rotary shaft 722. The distance between two adjacent rotating plates 721 in the axial direction may be constant or may be different as shown. In a case where the polymer PM and the depolymerization material are stirred and a part thereof is decomposed into a depolymerized product such as BHET, the viscosity or the degree of polymerization of the polymer PM decreases. Meanwhile, the closer to an inlet side (that is, the polymer feed port 711 side) of the container 710, the higher the viscosity or the degree of polymerization of the polymer PM is. Therefore, the polymer PM is more likely to adhere to the rotating plate 721. In a case where an interval between the rotating plates 721 on the inlet side of the container 710 is too small, the polymer PM adhering to each rotating plate 721 may interfere with each other, and there is a risk that the stirring (mechanical mixing with the depolymerization material) of the polymer PM and the rotation of the rotating body 720 is hindered. Therefore, as shown, it is preferable to reduce the interval between the rotating plates 721 from the polymer feed port 711 (preceding stage) of the container 710 toward the discharge port 712 (subsequent stage).

In this way, in the subsequent stage (the discharge port 712 side) where the interval between the rotating plates 721 is small, the polymer PM and the depolymerization material are efficiently stirred or mixed. Here, in a case where a plurality of the depolymerization material feed ports 714 are provided, the amount of the depolymerization material supplied from each of the depolymerization material feed ports 714 is increased from the preceding stage to the subsequent stage. In this manner, the efficiency of the mechanical mixing of the polymer PM and the depolymerization material can be further improved.

The rotating body 720 is rotationally driven by the rotation drive unit 740 configured by a motor or the like. Specifically, the rotation drive unit 740 is connected to the rotary shaft 722 of the rotating body 720, and rotationally drives the rotating body 720. In this way, in a case where the rotation drive unit 740 rotationally drives the rotary shaft 722, the plurality of rotating plates 721 fixed to the rotary shaft 722 integrally rotate. Then, the polymer PM supplied from the polymer feed port 711 to the container 710 and directed to the discharge port 712 is effectively mixed with the depolymerization material supplied to the container 710 from the depolymerization material feed port 714 by the plurality of rotating plates 721 which are rotating.

By performing the stirring by using the rotating body 720, the polymer PM and the depolymerization material are mechanically mixed, and also the decomposition (chemical reaction) into the depolymerized product such as BHET partially progresses. As a result, the IV value, the viscosity, and the degree of polymerization of the polymer PM such as PET in the container 710 gradually decrease as the polymer PM is moved from the polymer feed port 711 toward the discharge port 712. The position or height of the polymer PM adhering to each rotating plate 721 of the rotating body 720 indirectly represents the IV value, the viscosity, and the degree of polymerization of the polymer PM. Therefore, an adhesion position detection unit (not shown) may detect the highest reaching position of the polymer PM adhering to the rotating plate 721 in the vertical direction (Z direction) to identify the IV value, the viscosity, and the degree of polymerization of the polymer PM. For example, the adhesion position detection unit may detect the highest reaching position of the polymer PM adhering to the rotating plate 721 in the vertical direction (Z direction) through light advancing in a direction (Y direction) intersecting the vertical direction (Z direction) and the rotary shaft 722 (X direction).

A stirring mode adjusting unit (not shown) may adjust a stirring mode in the container 710 such that the deviation of the adhesion position of the polymer PM detected by the adhesion position detection unit (for example, the highest reaching position) from a desired position is reduced. The desired position here corresponds to the desired viscosity or the desired degree of polymerization of the polymer PM in the container 710. That is, the stirring mode adjusting unit may adjust the stirring mode in the container 710 such that the polymer PM in the container 710 has the desired viscosity or the desired degree of polymerization.

Specifically, as the stirring mode in the container 710, the stirring mode adjusting unit may adjust at least any one of the supply amount of the polymer PM and/or the depolymerization material to the container 710, the supply speed of the polymer PM and/or the depolymerization material to the container 710, the discharge amount to the outside of the container 710 through the discharge port 712, the discharge speed to the outside of the container 710 through the discharge port 712, the rotating speed of the rotating body 720, the pressure in the container 710, and the temperature in the container 710.

In addition to the adhesion position detection unit that indirectly detects the degree of polymerization of the polymer PM, a polymerization degree estimation unit (not shown) that estimates the degree of polymerization of the polymer PM based on the adhesion position of the polymer PM detected by the adhesion position detection unit may be provided. The stirring mode adjusting unit in this case adjusts the stirring mode in the container 710 such that the deviation of the degree of polymerization of the polymer PM estimated by the polymerization degree estimation unit from a desired value is reduced.

FIG. 13 schematically shows a horizontal type stirring device 384 as a fourth example of the mechanical mixing unit 380. The same components as those in FIG. 12 are denoted by the same reference numerals, and overlapping description is omitted. The horizontal type stirring device 384 constitutes the first depolymerization reaction tank 301 in FIG. 7. While the horizontal type stirring device 383 shown in FIG. 12 is a single-tank type, the horizontal type stirring device 384 shown in FIG. 13 is a multi-tank type (in the shown example, a two-tank type). Specifically, the horizontal type stirring device 384 includes a first horizontal type stirring device 384A on the preceding stage or upstream side and a second horizontal type stirring device 384B on the subsequent stage or downstream side. The first horizontal type stirring device 384A and the second horizontal type stirring device 384B in the shown example are both long in the X direction.

Both the first horizontal type stirring device 384A and the second horizontal type stirring device 384B include the above-described container 710, the rotating body 720, and the rotation drive unit 740. The discharge port 712 of the first horizontal type stirring device 384A is connected to the polymer feed port 711 of the second horizontal type stirring device 384B.

The rotating body 720 in the first horizontal type stirring device 384A and the second horizontal type stirring device 384B includes a plurality of rotating plates 721 separated from each other in the axial direction (X direction) of the rotary shaft 722. The distance in the axial direction between the adjacent rotating plates 721 in the first horizontal type stirring device 384A is constant, and the distance in the axial direction between the adjacent rotating plates 721 in the second horizontal type stirring device 384B is constant. Here, the constant distance between the adjacent rotating plates 721 in the second horizontal type stirring device 384B is preferably smaller than the constant distance between the adjacent rotating plates 721 in the first horizontal type stirring device 384A. In the second horizontal type stirring device 384B in the subsequent stage where the stirring of the polymer PM and the depolymerization material (and the decomposition of the depolymerized product such as BHET) progresses, the viscosity or the degree of polymerization of the polymer PM is lowered. Accordingly, the distance between the rotating plates 721 can be made smaller than that in the first horizontal type stirring device 384A.

In this way, in the second horizontal type stirring device 384B in the subsequent stage in which the interval between the rotating plates 721 is small, the polymer PM and the depolymerization material are efficiently stirred or mixed. Here, the amount of the depolymerization material supplied from the depolymerization material feed port 714 in the second horizontal type stirring device 384B is increased compared to the amount of the depolymerization material supplied from the depolymerization material feed port 714 in the first horizontal type stirring device 384A. In this manner, the efficiency of the mechanical mixing of the polymer PM and the depolymerization material can be further improved.

The present invention has been described above based on the embodiment. Various modification examples are possible in the combination of each component and each process in the embodiment as an example, and it is obvious to those skilled in the art that such modification examples are included within the scope of the present invention. In addition, the respective components of the embodiments above-described may be arbitrarily combined with each other as long as the respective operations and effects are not completely hindered.

In the example of FIG. 1, only one of each of the polymer adjustment device 200, the depolymerization reaction tank 300, the polymerization reaction tank 400, the by-product removal device 500, the polymer supply unit 600, and the like is provided, but a plurality of each of them may be provided. The plurality of processing units can execute the same processing in parallel, so that the processing performance of the processing unit group can be improved. In addition, the difference in the processing speed or the reaction rate between the processing units can be reduced by increasing the number of the slow processing units.

In addition, one or a plurality of the processing units may accept an external material that is procured from a location or a facility different from the chemical recycling molding system shown in FIG. 1 instead of or in addition to the material from the processing unit in the preceding stage. For example, in a case where a plurality of the polymerization reaction tanks 400 are provided, some of the polymerization reaction tanks 400 may be supplied with the depolymerized product from the depolymerization reaction tank 300, and the others may be supplied with the depolymerized product (synonymous with the depolymerized product supplied from a depolymerized product supply unit 300A which will be described later) procured from the outside. Similarly, in a case where a plurality of the by-product removal devices 500 and/or the polymer supply units 600 are provided, some of the by-product removal devices 500 and/or the polymer supply units 600 may be supplied with the polymer from the polymerization reaction tank 400, and the others may be supplied with the polymer (synonymous with the polymer supplied from a polymer supply unit 400A which will be described later) procured from the outside. In this way, by allowing the acceptance of the external material at each stage of the processing in the chemical recycling molding system, the flexibility of the chemical recycling molding system can be ensured, and the system can efficiently operate.

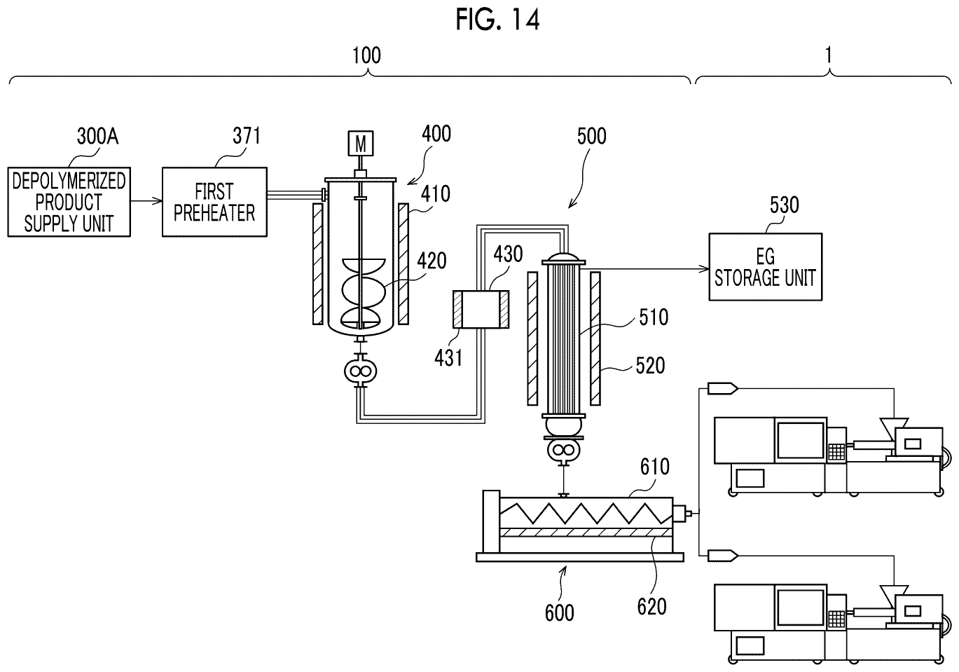

FIG. 14 shows a first modification example of the chemical recycling molding system. The same components as those in FIG. 1 are denoted by the same reference numerals, and overlapping description is omitted. In the present modification example, the depolymerized product supply unit 300A is provided instead of the depolymerization reaction tank 300 or the like in FIG. 1. The depolymerized product supply unit 300A supplies a depolymerized product such as BHET derived from the first molding product decomposed in a depolymerization reaction tank 300 (not shown) installed at a location or a facility different from the chemical recycling molding system shown in the drawing to the polymerization reaction tank 400.

A first preheater 371 that heats or preheats the depolymerized product such as BHET supplied from the depolymerized product supply unit 300A to a molten state may be provided in the subsequent stage of the depolymerized product supply unit 300A and the preceding stage of the polymerization reaction tank 400. The first preheater 371 may maintain the depolymerized product at the same temperature (between 220° C. and 250° C.) as that of the heating unit 320 provided in conjunction with the depolymerization reaction tank 300 provided in FIG. 1, or may maintain the depolymerized product at a temperature suitable for the polymerization reaction (between 250° C. and 300° C.), which is the same as that of the heating unit 410 provided in conjunction with the polymerization reaction tank 400.

In the polymerization reaction tank 400 and/or the by-product removal device 500, the EG as a by-product obtained together with the PET as a main product may be stored in an EG storage unit 530. The EG stored in the EG storage unit 530 may be used for other purposes at other locations or facilities, or may be sold to a consumer.

In the first modification example as described above, the polymer resynthesized in the polymerization reaction tank 400 is not made into flakes or pellets, but is supplied as it is to the injection molding machine 1 by the polymer supply unit 600. Since the cooling process and the heating process related to the flakes or the pellets as in the related art are not required, the molding product such as a PET bottle can be recycled with less energy as compared with the related art.

In the chemical recycling device 100 according to the first modification example, since the polymer resynthesized in the polymerization reaction tank 400 is supplied as it is to the injection molding machine 1, it is necessary to quickly realize the IV value of the polymer required for the molding product (second molding product). In the present modification example, the by-product removal device 500 having a function of promoting the polymerization reaction and increasing the IV value of the polymer is provided in addition to the polymerization reaction tank 400. Therefore, the by-product removal device 500 can sufficiently meet such a requirement.

Although not shown, the polymer return unit 900 similar to that of another embodiment (FIG. 4) may be provided in the first modification example. This polymer return unit 900 returns at least a part of the polymer synthesized in the polymerization reaction tank 400 to the polymerization reaction tank 400 instead of sending the polymer to the polymer supply unit 600 at least in an unacceptable period during which the injection molding machine 1 cannot accept the total amount of the polymer from the polymer supply unit 600.

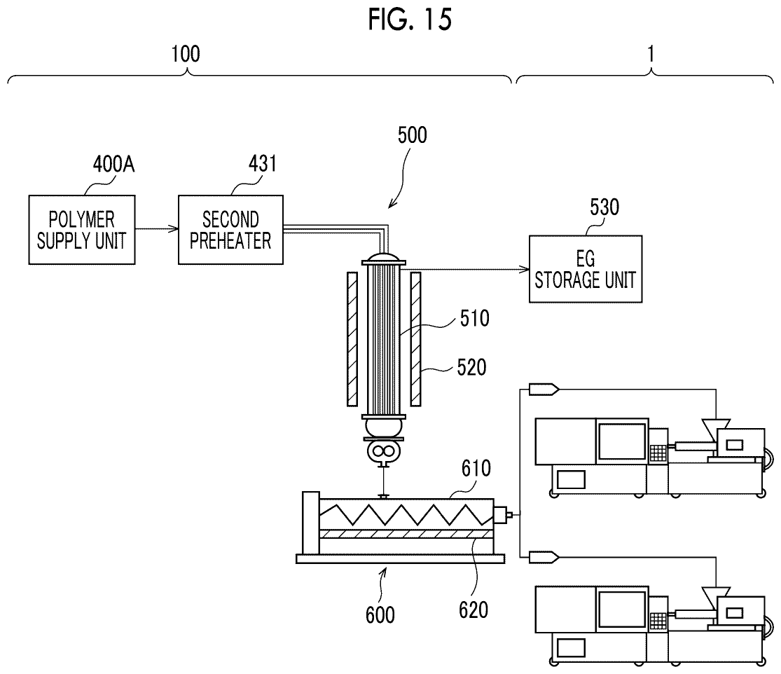

FIG. 15 shows a second modification example of the chemical recycling molding system. The same components as those in FIG. 1 and/or FIG. 14 are denoted by the same reference numerals, and overlapping description is omitted. In the present modification example, the polymer supply unit 400A is provided instead of the depolymerization reaction tank 300 and the polymerization reaction tank 400 in FIG. 1. The polymer supply unit 400A supplies the polymer such as PET derived from the first molding product synthesized in a polymerization reaction tank 400 (not shown) installed at a location or facility different from the chemical recycling molding system shown in the drawing to the by-product removal device 500 and/or the polymer supply unit 600.

A second preheater 431 that heats or preheats the polymer such as PET supplied from the polymer supply unit 400A to a molten state may be provided in the subsequent stage of the polymer supply unit 400A and in the preceding stage of the by-product removal device 500 and/or the polymer supply unit 600. The second preheater 431 may maintain the polymer at the same temperature (between 250° C. and 300° C.) as that of the heating unit 410 that is provided in conjunction with the polymerization reaction tank 400 provided in FIG. 1 or FIG. 14, may maintain the polymer at a suitable temperature (between 250° C. and 290° C.) for the polymerization reaction, which is the same as that of the heating unit 520 that is provided in conjunction with the by-product removal device 500, or may maintain the polymer at the same temperature (between 250° C. and 290° C.) as that of the heating unit 620 that is provided in conjunction with the polymer supply unit 600.

The EG obtained together with the PET in the by-product removal device 500 may be stored in the EG storage unit 530. The EG stored in the EG storage unit 530 may be used for other purposes at other locations or facilities, or may be sold to a consumer.

In the chemical recycling device 100 according to the second modification example, since the polymer supplied from the polymer supply unit 400A is supplied as it is to the injection molding machine 1, it is necessary to quickly realize the IV value of the polymer required for the molding product (second molding product). In the present modification example, the by-product removal device 500 having a function of promoting the polymerization reaction and increasing the IV value of the polymer is provided. Therefore, the by-product removal device 500 can sufficiently meet such a requirement.

The configurations, operations, and functions of each device and each method described in the embodiment can be realized by hardware resources or software resources, or by the cooperative operation of hardware resources and software resources. For example, a processor, a ROM, a RAM, and various integrated circuits can be used as the hardware resources. For example, program such as an operating system and applications can be used as the software resources.

The present invention relates to a chemical recycling device and the like.

It should be understood that the invention is not limited to the above-described embodiment, but may be modified into various forms on the basis of the spirit of the invention. Additionally, the modifications are included in the scope of the invention.

What is claimed is:

1. A chemical recycling device comprising:
a depolymerization reaction tank that decomposes a first molding product made of a polymer into a depolymerized product through a depolymerization reaction;
a polymerization reaction tank that synthesizes the depolymerized product into the polymer through a polymerization reaction; and
a polymer supply unit that supplies the polymer synthesized in the polymerization reaction tank to a molding machine that molds a second molding product.

2. A chemical recycling device comprising:
a polymerization reaction tank that synthesizes a depolymerized product obtained by decomposing a first molding product made of a polymer through a depolymerization reaction into the polymer through a polymerization reaction; and
a polymer supply unit that supplies the polymer synthesized in the polymerization reaction tank to a molding machine that molds a second molding product.

3. A chemical recycling device comprising:
a polymer supply unit that supplies a polymer, which is synthesized through a polymerization reaction from a depolymerized product obtained by decomposing a first molding product made of the polymer through a depolymerization reaction, to a molding machine that molds a second molding product.

4. The chemical recycling device according to claim 1,
wherein a by-product removal device that removes a by-product generated together with the polymer through the polymerization reaction in the polymerization reaction tank is provided between the polymerization reaction tank and the polymer supply unit.

5. The chemical recycling device according to claim 4,
wherein the polymer is polyethylene terephthalate, the depolymerized product is bis(2-hydroxyethyl) terephthalate, and the by-product is ethylene glycol.

6. The chemical recycling device according to claim 1,
wherein the polymer supply unit supplies the polymer synthesized in the polymerization reaction tank to the molding machine in a molten state.

7. The chemical recycling device according to claim 6,
wherein the polymer supply unit includes a first heating unit that heats the polymer synthesized in the polymerization reaction tank to maintain the polymer in a molten state.

8. The chemical recycling device according to claim 7,
wherein a by-product removal device that removes a by-product generated together with the polymer through the polymerization reaction in the polymerization reaction tank is provided between the polymerization reaction tank and the polymer supply unit, a second heating unit that heats the polymer synthesized in the polymerization reaction tank and that maintains the polymer in a molten state is provided in the polymerization reaction tank, the by-product removal device, and/or therebetween, and a first heating temperature by the first heating unit is higher than a second heating temperature by the second heating unit.

9. The chemical recycling device according to claim 1,
wherein the polymer is polyethylene terephthalate, and the depolymerized product is bis(2-hydroxyethyl) terephthalate.

10. The chemical recycling device according to claim 1,
wherein the depolymerized product includes a monomer of the polymer.

11. The chemical recycling device according to claim 1,
wherein the first molding product and the second molding product are the same type.

12. The chemical recycling device according to claim 11,
wherein the first molding product and the second molding product are PET bottles.

13. The chemical recycling device according to claim 1,
wherein the molding machine is an injection molding machine.

14. The chemical recycling device according to claim 2,
wherein a first preheater that heats the depolymerized product before being supplied to the polymerization reaction tank is provided.

15. The chemical recycling device according to claim 3,
wherein a second preheater that heats the polymer before being supplied to the polymer supply unit is provided.

16. The chemical recycling device according to claim 1, further comprising:
a polymer return unit that returns at least a part of the polymer synthesized in the polymerization reaction tank to the polymerization reaction tank.

17. The chemical recycling device according to claim 1, further comprising:
a polymer return unit that returns at least a part of the polymer synthesized in the polymerization reaction tank to the depolymerization reaction tank.

18. The chemical recycling device according to claim 1, further comprising:
a polymer return unit that returns at least a part of the polymer synthesized in the polymerization reaction tank to the polymerization reaction tank and the depolymerization reaction tank.

19. The chemical recycling device according to claim 18,
wherein the polymer return unit returns more of the polymer to the polymerization reaction tank than to the depolymerization reaction tank at least before an unacceptable period during which the molding machine cannot accept a total amount of the polymer from the polymer supply unit exceeds a predetermined period threshold, and returns more of the polymer to the depolymerization reaction tank than to the polymerization reaction tank after the unacceptable period exceeds the period threshold.

20. The chemical recycling device according to claim 1,
wherein the depolymerization reaction decomposes the polymer into the depolymerized product by using a depolymerization material, and the depolymerization reaction tank includes a mechanical mixing unit that mechanically mixes the depolymerization material and the polymer at least in an early stage of the depolymerization reaction.
